(19) United States
(12) Patent Application Publication
(10) Pub. No.: US 2021/0212918 A1
(43) Pub. Date: Jul. 15, 2021

(54) SELF-CURING ACRYLIC COMPOSITION FOR HAIR STYLING

(71) Applicant: EASYFIX HAIR DESIGN LTD., Tel Aviv (IL)

(72) Inventors: Moshe UZAN, Petah Tikva (IL); Steve DAREN, Nes Ziona (IL)

(21) Appl. No.: 15/734,462

(22) PCT Filed: Jun. 3, 2019
(86) PCT No.: PCT/IL2019/050628

§ 371 Date: Dec. 2, 2020

(30) Foreign Application Priority Data
Jun. 3, 2018 (IL) 259785

(51) Int. Cl. A61K 8/81; A61K 8/86; A61Q 5/04; A45D 7/06

(52) U.S. Cl. CPC A61K 8/8152; A61K 8/8147; A45D 7/06; A61Q 5/04; A61K 8/86

(57) ABSTRACT

The present invention provides hair straightening compositions comprising acrylic polymers configured to crosslink upon drying and thereby to form a film that is able to preserve hair in straightened conformation. Also provided is a hair straightening cosmetic formulation comprising said hair straightening composition and its use in straightening hair or preserving hair in a straightened conformation. The hair straightening effect is temporary and can be removed when the formulation is washed away in water or with shampoo. The cosmetic formulations of the present invention are ready to use formulations and may be applied frequently.

### FIELD OF THE INVENTION

The present invention relates to hair treatment compositions comprising an acrylic emulsion comprising acrylic based polymers that undergo crosslinking upon drying, in the form of hair-dressing compositions comprising a cosmetically acceptable medium and a hair treatment process using same.

### BACKGROUND OF THE INVENTION

Women of all ages often prefer to style their hair long and straight. The majority of women, however, do not have such hair but rather wavy, curly or dispersed one. Different hair styling procedures are known to straighten curly hair. Previously known processes used to straighten curly hair are both expensive and damaging to the hair fibers and therefore may not be used more than at intervals of several months or more. Known procedures of hair straightening involve changing the hair chemistry by attacking the sulfur-sulfur bonds in keratin, the main molecule in hair.

With a keratin treatment commonly known as the Brazilian Technique, the protein keratin formula is infused with an iron into freshly washed hair using intense heat, thus only impacting the outside layer of the hair. This procedure involves reacting the keratin with the toxic chemical formaldehyde. This procedure is banned in many countries. The second procedure, known as the Japanese procedure or thermal reconditioning, involves a chemical treatment and then heating the hair to a high temperature with an iron. Japanese hair straightening works by changing the inner bonds of hair. This causes the keratin to start to decompose and the hair can be straightened. This procedure is expensive, time-consuming, and may cause long term damage to the hair.

Use of acrylic polymers, also referred to as acrylates, is well known in many fields and industries such as the paint and coating industry, cosmetics, medicine and agriculture. This use is further exemplified by U.S. Pat. No. 4,217,396.

The paints and coatings industry relies heavily on water-based acrylic emulsions. To aid in the formation of water and solvent-resistant solid coatings, self-cross-linking polymeric dispersants have been developed. In such systems, all of the reactants for cross-linking are present in the emulsion and may be stable for many years. The cross-linking may be triggered by the evaporation of the water, change of pH or curing at elevated temperatures. Photo-curing is also an option. The incorporation of crosslinking chemistry in waterborne coatings or film forming compositions is recognized to provide a particularly effective means of enhancing the mechanical strength, chemical stability and solvent resistance of the final film. This process of coating formation is described among others by Buckmann et al. (European Coatings Journal, 2001, 6, 53-60). Film-forming acrylic polymers have even been used to increase storage stability of cut flowers as described in U.S. Pat. No. 8,664,161.

In the cosmetics and personal-care industries, many formulations of shampoos, creams and conditioners utilize water-based acrylic emulsions. These are considered to be safe and reliable. Acrylate copolymers such as Luvimer® (BASF), Balance® (Akzo Nobel) or Acudyne® (Rohm and Haas) are used in the hair care industry. Use of emulsions of acrylic polymers for hair styling is described for example in U.S. Pat. Nos. 9,249,247, 9,345,656 and 9,717,673. U.S. Pat. No. 9,365,739 describes waterborne two component polyurethane coating compositions comprising alicyclic rigid polyols further comprising a vinyl or acrylic emulsion polymer. WO 2013/103528 describes polymers containing an acetoacetate moiety.

There exists a need and a market for a user-friendly formulation applied to curly or wavy hair, preferably at home but also in a hair salon, that can be applied at frequent intervals for temporary straightening of the hair. The formulation should be applied at moderate temperatures and without the use of toxic chemicals that alter the hair structure chemically.

### SUMMARY OF THE INVENTION

Compositions and method for the temporary or semi-permanent styling, revitalization, straightening, and optionally tinting of fibers, and in particular human hair, by applying to the hair fibers compositions containing a blend of acrylic polymers in personal care products are provided. It is disclosed that on the hair surface, the polymers are converted to a cross-linked film that is stable for hours or days which maintains styling and which protects the hair against humidity. The hair is surrounded by a protective envelope and is consequently protected from external aggression. The envelope is able to preserve the styling even under harsh conditions such as high humidity. The blend is suitable for use in cosmetic applications for mammalian hair, e.g. human hair, equine manes and tails and pet coats. Advantageously, the compositions and formulations of the present invention achieve the straightening effect without causing any chemical reaction or damage to the hair.

It has been surprisingly found that self-cross-linking acrylic polymers when formulated in hair care compositions can create a restraining coating around individual or groups of hair that enable them to maintain the hair's shape, e.g. maintaining hair's wet shape even after the hair has been dried. This shape is maintained until the hair is next shampooed or washed.

Without being bound to any particular theory, it is assumed that the coating is obtained by formation of a film around hairs upon drying. Such a film-forming process can be described as proceeding in three stages:
Stage 1: As the water evaporates, the polymer emulsion particles come into close contact and start to agglomerate;
Stage 2: Particle deformation and packing; and
Stage 3: Polymer coalescence—chains entangle and react (cross-link with other polymers) and particle boundaries disappear. At this point an essentially continuous film is formed.

The coating film around hair, formed by the polymers of the compositions of the present invention, provides a much better and longer-lasting hair styling than any known hair styling product utilizing acrylic polymers. Formation of the enhanced film is conferred by cross-linkers present within the polymers. The cross-linkers covalently bind polymers to form a polymeric net. The resulting film allows maintaining hair, e.g. in straightened conformation, for a prolonged period even under humid conditions. On the other hand, the film can be removed when washed or shampooed in water.

In one aspect, the present invention provides a hair straightening composition comprising from about 5 wt % to about 55 wt % of at least one acrylic-based polymer comprising acrylic units that act as cross-linkers that will form bonds between the polymer chains wherein the amount of said cross-linker units is from about 1% to about 25 wt % of said acrylic-based polymer and from about 0.5 wt % to about 10 wt % of a non-ionic surfactant, wherein the non-ionic surfactant is selected from a pharmaceutical or cosmetic grade surfactant, and wherein the polymers undergo cross linking and thereby maintain the hair in a straightened configuration upon drying.

According to some embodiments the polymer is derived from (meth)acrylic monomers and/or (meth)acrylic oligomers such as monoacrylate oligomer, isobornyl acrylate, butyl acrylate, lauryl methacrylate, and any combination thereof. According to one embodiment, the polymer is derived from a combination of (meth)acrylic monomers and acrylate containing oligomers. According to one embodiment, the polymer is derived form a combination of monoacrylate oligomer and isobornyl acrylate. According to another embodiment, the polymer is derived form a combination of monoacrylate oligomer, isobornyl acrylate and butyl acrylate. According to one embodiment, the polymer is derived form a combination of monoacrylate oligomer, isobornyl acrylate and lauryl methacrylate. In other words, the polymer comprises the units (the moieties) of the monomers from which it is derived. According to some embodiments the cross-linker unit is 2-(methacryloyloxy)ethylacetoacetate (AAEM). According to some embodiments, the non-ionic surfactant is selected from the group consisting of ethoxylated and/or propoxylated alcohol or ester, tyloxapol, polysorbate (TWEEN), PPG-5-ceteth-20, PEG-20 sorbitan isostearate, alkoxylated alcohols, alkoxylated polyol esters, decyl glucoside, coco-glucoside, sucrose laurate, sorbitol, polyoxyethylene, sorbitan monolaurate, Laureth comprising from 20 to 40 units of ethylene oxide such as Laureth-20, Laureth-21, Laureth-25, Laureth-30, Laureth-38, Laureth-40, Disponil AFX 4060 (modified ethoxylated fatty alcohols polyoxyethylene 40) and Sabowax SE40 (polyethylene glycol-40 stearate) and any combination hereof. According to some embodiments, the hair straightening composition of the present invention comprises from about 2 wt % to about 15 wt % of said surfactant.

According to a particular embodiment, the hair straightening composition of the present invention comprises from about 15 wt % to about 30 wt % of said polymer, a cross-linker in the amount of about 2 wt % to about 20 wt % of said polymer, and from about 3 wt % to about 10 wt % of said non-ionic surfactant. The composition of the present invention may comprise from about 20 wt % to about 50 wt % solids. According to some embodiments, the glass transition temperature (Tg) of the polymer is from about 10° C. to about 50° C. According to other embodiments, the polymers of the composition of the present invention undergo crosslinking upon drying.

According to another aspect, the present invention provides a hair straightening formulation for application to the hair of a subject comprising from about 2 wt % to 40 wt % of the hair straightening composition of the present invention. According to some embodiments, the hair straightening formulation is in the form of a spray, lotion, cream, foam, mousse, or gel. The formulation of the present invention maintains or preserve straightened hair straight conformation upon drying.

According to some embodiments, the formulation of the present invention further comprises cosmetically acceptable ingredients selected from the group consisting of preservatives, thickeners, viscosity modifiers, fatty alcohols, pH adjusting agents, buffers, coloring agents, guar gums, perfumes, PEGs, essential oils, silicone fluid dimethicone, hydroxypropyl cellulose, carboxymethyl cellulose and any combination thereof.

According to another embodiment, the present invention provides a method of straightening hair comprising applying the hair straightening formulation of the present invention to hair and drying the formulation. According to some embodiments, the method further comprises the step of straightening the hair by combing or brushing before drying. According to some embodiments, the formulation of the present invention is applied to dry or wet hair. According to another embodiment, the formulation is dried by gentle heat or at room temperature.

### BRIEF DESCRIPTION OF DRAWINGS

FIG. 1 shows the schematic presentation of the acrylic polymer of the present invention in water suspension (upper panel) and after drying and crosslinking (lower panel).

FIG. 2 shows a scheme of polymeric network on the hair surface before (left panel) after combing (Hair arrangement) (right panel).

FIG. 3 shows the hair after application of 1 g of Formulations 64-2, 64-19, 65-2 and 65-19.

FIG. 4 shows the hair after application of Formulations 62-B, 62-2, 62-19, 63-2, 63-19, 64-2, 64-19, 65-2 and 65-19 (from left to right) and drying.

FIG. 5 shows the hair treated with a formulation of the present invention without any composition of the present invention (the most left strand) or with a complete formulation including the compositions 2-18-2 and 2-18-19, respectively (the two right strands).

FIG. 6 shows typical untreated swatches of Brazilian hair type 2.

FIG. 7 shows swatches of Brazilian hair type 2 after treatment with formulations 101 (swatch no. 1), with formulation 105 (swatch no. 2) or with water (swatches 3-5) (FIG. 7A) and after 3 days in humid conditions (FIG. 7B).

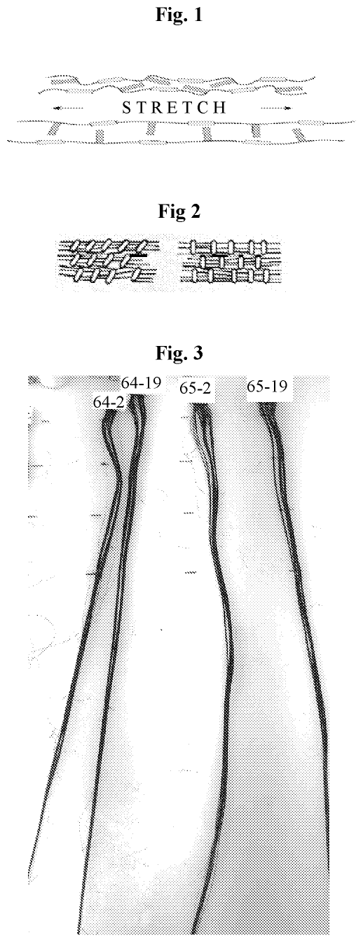

### DETAILED DESCRIPTION OF THE INVENTION

The present invention relates to a composition for preserving straightened hair in an elongated form and conferring the hair smoothness, promoting a lustrous shine and have a good mending effect. The composition of the present invention allows the reduction of the total volume of curly and frizzy hair. The compositions of the present invention provides also protection against humidity and other ambient stresses.

According to one aspect, the present invention provides a hair styling composition comprising from about 5 wt % to about 55 wt % of at least one acrylic-based polymer comprising acrylic units that act as cross-linkers that will form bonds between the polymer chains and from about 0.5 wt % to about 15 wt % of a non-ionic surfactant, wherein the amount of said cross-linker units is from about 1% to about 25 wt % of said acrylic-based polymer and the non-ionic surfactant is selected from a pharmaceutical or cosmetic grade surfactant, and wherein the composition undergoes cross linking and thereby straightens the hair upon drying. According to one embodiment, hair styling is hair straightening. Thus in one aspect, the present invention provides a hair straightening composition comprising from about 5 wt % to about 55 wt % of at least one acrylic-based polymer comprising acrylic units that act as cross-linkers wherein the amount of said cross-linker units is from about 1% to about 25 wt % of said acrylic-based polymer, and from about 0.5 wt % to about 15 wt % of a non-ionic surfactant selected from a pharmaceutical or cosmetic grade surfactant, and wherein the polymers undergo cross linking and thereby straighten the hair upon drying. According to one embodiment, the cross-linkers form bonds between the polymer chains upon drying of the composition.

The term “hair straightening” encompasses also the meaning of preserving or maintaining the straightened hair straight, e.g. maintaining hair straightened after wetting the hair. Thus the terms “hair straightening”, “preserving hair in straight conformation”, “preserving hair in straightened conformation”, “maintaining hair straight”, “maintaining the hair straightened” and “maintaining straightened hair straight” may be used herein interchangeably. According to one embodiment, the length of the straightened hair upon drying the composition is up to 200%, up to 160% or up to 140% of the hair's length in un-straightened conformation. According to another embodiment, the length of the straightened hair upon drying the composition is from 101% to 130%, 105% to 125%, or from 110% to 120% of the hair's length in un-straightened conformation. The un-straightened conformation means the conformation of dry and untreated hair. In one embodiment, the un-straightened conformation comprises curly, wavy or kinky hair. According to some embodiments, straight or straightened conformation do not necessarily means that the hair is perfectly straight, but rather that the hair is longer and/or straighter than its initial state.

As used herein, the terms “composition”, “emulsion”, “stock composition”, “stock emulsion” and “polymer emulsion” can be used interchangeably.

As used herein, the terms “polymer” and “polymer chain” are used interchangeably and refer to a single polymeric chain that did not undergo crosslinking with another polymeric chain. According to the teaching of the present invention, polymer chains upon crosslinking form a network of polymers—a film.

According to some embodiments, the composition of the present invention temporarily preserves hair in the straightened conformation. According to another embodiment, the composition of the present invention preserves or maintains hair in straightened conformation from several hours to several days. According to one embodiment, the composition of the present invention preserves hair in the straightened conformation for from 1 to 24, from 3 to 20, from 4 to 16, or from 6 to 12 hours, provided that that hair is not wetted, washed or shampooed. According to one embodiment, the composition of the present invention preserves hair in the straightened conformation for 1 to 5, from 2 to 4, or for 3 days, provided that that hair is not wetted, washed or shampooed. According to other embodiments, the composition of the present invention preserves hair in the straightened conformation for 3 to 14, for 5 to 12 or for 7 to 10 days.

According to one embodiment, the hair straightening composition comprises from about 5 wt % to about 50 wt % of acrylic-based polymers. According to one embodiment, the hair straightening composition comprises from about 5 wt % to about 45 wt % or about 10 to about 40 wt % of acrylic-based polymers. According to another embodiment, the composition comprises from about 15 wt % to about 35 wt % of acrylic-based polymers. According to yet another embodiment, the composition comprises from about 20 wt % to about 35 wt % of acrylic-based polymers. According to another embodiment, the composition comprises from about 20 wt % to about 40 wt %, from about 22 to about 38 wt %, from about 25 wt % to about 35 wt %, or from about 28 wt % to about 32 wt % of acrylic-based polymers. According to one embodiment, the composition comprises from about 10 wt % to about 30 wt % or from 15 to 25 wt % of acrylic-based polymers.

According to another embodiment, the polymer comprises from about 2 wt % to about 25 wt % of acrylic-based cross-linker units. According to another embodiment, the polymer comprises from about 2.5 wt % to about 20 wt % of the cross-linker. According to some embodiments, the polymer comprises from about 2.5 to 18 wt %, from 3 to 17 wt %, from 3.5 to 16 wt %, from 4 to 15 wt % from 4.5 to 14 wt %, from 5 to 12 or from 7 wt % to about 18 wt % of the cross-linkers. According to some embodiments, the polymer comprises from about 10 wt % to about 15 wt % of the cross-linker units. According to certain embodiments, the amount of the cross-linker units is from about 6% to about 20 wt % of said acrylic-based polymer. According to one embodiment, the amount of the cross-linker units is from about 8% to about 18 wt % of said acrylic-based polymer. According to some embodiments, the amount of the cross-linker units is from about 2% to about 10 wt % of said acrylic-based polymer. According to one embodiment, the amount of the cross-linker units is from about 3 to about 9 wt % of said acrylic-based polymer. According to another embodiment, the amount of said cross-linker units is from about 4% to about 8 wt % of said acrylic-based polymer. According to some embodiments, the amount of the cross-linker units is about 6, about 8, about 10, about 12, about 13, about 14, about 15, about 16, about 17, about 18, about 19 or about 20 wt % of said acrylic-based polymer. According to some embodiments, the composition comprises from 1 to 10 wt % of the cross-linker units. According to other embodiments, the composition comprises from 2 to 8 wt % of the cross-linkers. According to one embodiment, the composition comprises from 2 to 6 wt % of the cross-linkers. According to another embodiment, the composition comprises from 3 to 8 wt % of the cross-linkers.

According to some embodiments, the composition comprises from about 20 wt % to about 40 wt %, from about 22 to about 38 wt %, from about 25 wt % to about 35 wt %, or from about 28 wt % to about 32 wt % of acrylic-based polymers and the polymer comprises from about 5 wt % to about 20 wt % of the cross-linker or from about 7 wt % to about 18 wt % of the cross-linker.

According to any one of the above embodiments, the composition comprises from about 0.5 to about 15 wt % or from 2 wt % to about 10 wt % of non-ionic surfactant. According to certain embodiments, the composition comprises from about 0.5 to about 10 wt % of non-ionic surfactant. According to one embodiment, the composition comprises from about 1 wt % to about 9 wt %, from about 2 wt % to about 8 wt %, from about 3 to about 7 wt % of the non-ionic surfactant. According to one embodiment, the composition comprises from about 4 wt % to about 6 wt % of a non-ionic surfactant.

According to some embodiments, the composition comprises from about 20 wt % to about 40 wt %, from about 22 wt % to about 38 wt %, from about 25 wt % to about 35 wt %, or from about 28 wt % to about 32 wt % of acrylic-based polymers, from about 2 wt % to about 8 wt % non-ionic surfactant and the polymer comprises from about 5 wt % to about 20 wt % of the cross-linker or from about 7 wt % to about 18 wt %, or from about 3 to about 10 wt % of the cross-linker units.

According to one embodiment, the present invention provides a hair straightening composition comprising from about 5 wt % to about 55 wt % of at least one acrylic-based polymer comprising acrylic-based cross-linker units and from about 0.5 wt % to about 8 wt % of a non-ionic surfactant, wherein the amount of said cross-linkers is from about 2.5 to about 25 wt % of said acrylic-based polymer and the non-ionic surfactant is selected from a pharmaceutical or cosmetic grade surfactant, and wherein the composition straightens the hair upon drying. According to another embodiment, the composition comprises from about 15 wt % to about 30 wt % of said polymer comprising cross-linker units in the amount of about 5 wt % to about 20 wt % of said polymer, and from about 3 wt % to about 10 wt % of said non-ionic surfactant.

According to some of the above embodiments, the acrylic-based polymer is derived from acrylic monomers. According to other of the above embodiments, the acrylic-based polymer is derived from methacrylic monomers. According to other of embodiments, the acrylic-based polymer is derived from acrylic oligomers. According certain embodiments, the acrylic-based polymer is derived from a mixture of (meth)acrylic monomers and acrylic oligomers such as monoacrylic oligomers. According to some embodiments, the acrylic-based polymer is derived from a mixture of acrylic monomers such as acrylic monomer, methacrylic monomers and a combination thereof, and monoacrylic oligomers. The term “acrylic-based polymer”, as used herein has the meaning that at least 50 wt % of the monomers moieties forming said polymer are acrylic moieties. The term “derived” has the meaning of obtained from said monomers via a chemical reaction, i.e. via polymerization of said monomers. It is contemplated by the teaching of the present invention that the polymer may contain non-acrylic monomer moieties. According to any one of the above embodiment, the content of acrylic units that form the polymer is from 85 to 100 wt % of said polymer. Thus, according to one embodiment, the polymer comprises from 85 to 100 wt %, from 90 to 98 wt %, from 95 to 97 wt % or from 95 to 99 wt % of acrylic moieties. According to one embodiment, the cross-linker unit and acrylic monomer and/or acrylic oligomers moieties collectively constitute from 85 to 100% of the polymer. According to another embodiment, the cross-linker unit and acrylic monomer and/or acrylic oligomers moieties collectively constitute from 90 to 99 wt % from 90 to 98 wt %, 95 to 97 wt % or from 95 to 99 wt % of the polymer. According to some embodiments, the polymer consists essentially of acrylic based units and acrylic-based cross linker units. According to other embodiments, the polymer consists of acrylic moieties and acrylic-based cross-linker moieties.

The terms “acrylic monomers” and “acrylate monomers” are used herein interchangeably and refer to monomers based on the structure of acrylic acid and/or methacrylic acid. The term “acrylic monomers” also encompasses methacrylate monomer. The term “acrylic oligomer” as used herein refers to oligomers comprising an acrylic or methacrylic group. According to one embodiment, the acrylic monomers are selected from the group consisting of isobornyl acrylate, butyl acrylate, lauryl methacrylate, and any combination thereof. According to one embodiment, the acrylic monomer is any alkyl acrylate. The term “alkyl” as used herein typically means a straight or branched saturated hydrocarbon radicals having 1-18 carbon atoms and includes, e.g., methyl, ethyl, n-propyl, isopropyl, n-butyl, sec-butyl, isobutyl, tert-butyl, n-pentyl, 2,2-dimethylpropyl, and the like. According to other embodiments, the acrylic monomer is selected from methacrylate, isobornyl methacrylate, butyl methacrylate, lauryl methacrylate, According to one embodiment, the acrylic monomer is any alkyl methacrylate. According to some embodiments, acrylic oligomer is monoacrylate oligomer. As such, the term oligomer refers to the side chains of the monoacrylate. According to one embodiment, the monoacrylate oligomer is 2-hydroxy-3-(prop-2-enoyloxy)propyl 2-methyl-2-propylhexanoate (CN152 of Sartomer having CAS number 444649-70-1). According to another embodiment, the monoacrylate oligomer is selected from polyester mono-(meth)acrylate, urethane mono-(meth)acrylate and amine-modified mono-(meth)acrylate.

According to one embodiment, the polymer is derived from a combination of monoacrylate oligomer and isobornyl acrylate. According to one embodiment, the weight ratio between monoacrylate oligomer and isobornyl acrylate is from 1:10 to 10:1, from 1:9 to 9:1, from 1:8 to 8:1, from 1:7 to 7:1, from 1:6 to 6:1, from 1:5 to 5:1, from 1:4 to 4:1, from 1:3 to 3:1 or from 1:2 to 2:1. According to certain embodiments, the weight ratio between monoacrylate oligomer and isobornyl acrylate is 1.5:1 to 1:1.5. According to one embodiment, the monoacrylate oligomer is 2-hydroxy-3-(prop-2-enoyloxy)propyl 2-methyl-2-propylhexanoate.

According another embodiment, the polymer is derived from a combination of monoacrylate oligomer, isobornyl acrylate and butyl acrylate. According to one embodiment, the weight ratio between monoacrylate oligomer, isobornyl acrylate and butyl acrylate is from 10:10:1 to 1:1:10 or about from 10:9:2. According to another embodiment, the weight ratio between monoacrylate oligomer, isobornyl acrylate and butyl acrylate is from 10:10:1 to 1:10:1. According to a further embodiment, the weight ratio between monoacrylate oligomer, isobornyl acrylate and butyl acrylate is from 10:10:1 to 10:1:1. According to a certain embodiment, the weight ratio between monoacrylate oligomer, isobornyl acrylate and butyl acrylate is from 10:10:1 to 10:10:10. According to one embodiment, the monoacrylate oligomer is 2-hydroxy-3-(prop-2-enoyloxy)propyl 2-methyl-2-propylhexanoate.

According to yet another embodiment, the polymer is derived from a combination of monoacrylate oligomer, isobornyl acrylate and lauryl methacrylate. According to one embodiment, the weight ratio between monoacrylate oligomer, isobornyl acrylate and lauryl methacrylate is from 10:10:1 to 1:1:10 or about from 10:9:2. According to another embodiment, the weight ratio between monoacrylate oligomer, isobornyl acrylate and lauryl methacrylate is from 10:10:1 to 10:1:1. According to one embodiment, the weight ratio between monoacrylate oligomer, isobornyl acrylate and lauryl methacrylate is from 10:10:1 to 1:10:1. According to a certain embodiment, the weight ratio between monoacrylate oligomer, isobornyl acrylate and lauryl methacrylate is from 10:10:1 to 10:10:10. According to one embodiment, the monoacrylate oligomer is 2-hydroxy-3-(prop-2-enoyloxy)propyl 2-methyl-2-propylhexanoate.

According to any one of the above embodiments, the polymer comprises the units (the moieties) of the monomers from which it is derived. According to some embodiments, the ratios of the monomer units within the polymer are present at the ratios as defined hereinabove.

According to any one of the above embodiment, the cross-linker is 2-(methacryloyloxy)ethylacetoacetate (AAEM) or a derivative thereof. According to another embodiment, the cross-linker is selected from N-methylol acrylamide, methacrylic acid derivative, azridenes with acid groups; amines with epoxy(oxirane); alkoxy silanes; n-methylol acrylamide; and diacetone acrylamide dihydrazide. The terms “cross-linker”, “cross-linker unit”, “acrylic cross-linker” and “acrylic cross-linker unit” are used herein interchangeably and include both bifunctional and multifunctional units. According to the teaching of the present invention, cross-linkers are parts of a polymer chain and are capable of binding (cross-linking) to another polymer chain thereby producing a net or a film of polymers. According to one embodiment, the polymer upon drying and crosslinking forms a film around hair. Thus according to any one of the embodiments, the present invention provides a hair straightening composition comprising from about 5 wt % to about 55 wt % of at least one acrylic-based polymer comprising acrylic units that act as cross-linkers capable of forming bonds between the polymers and from about 0.5 wt % to about 15 wt % of a non-ionic surfactant, wherein the amount of said cross-linker units is from about 2.5% to about 25 wt % of said acrylic-based polymer and the non-ionic surfactant is selected from a pharmaceutical or cosmetic grade surfactant, and wherein the polymer chains undergo cross linking and thereby straightens the hair upon drying. According to another embodiment, the present invention provides a hair straightening composition comprising from about 5 wt % to about 55 wt % of at least one acrylic-based polymer comprising acrylic units that act as cross-linkers and from about 0.5 wt % to about 15 wt % of a non-ionic surfactant, wherein the amount of said cross-linker units is from about 2.5% to about 25 wt % of said acrylic-based polymer and the non-ionic surfactant is selected from a pharmaceutical or cosmetic grade surfactant, and wherein the polymer chains undergo cross linking via said cross-linking units and thereby straightens the hair upon drying, e.g. maintain the straightened hair in straight conformation.

According to any one of the above embodiments, composition comprises a non-ionic surfactant. According to one embodiment, the non-ionic surfactant is selected from the group consisting of ethoxylated and/or propoxylated alcohol or ester, polyoxyether, Disponil AFX 4060 (fatty acid alcohol polyglycol ether or more specifically modified ethoxylated fatty alcohols polyoxyethylene (POE) 40), Sabowax SE40 (polyethylene glycol-40 stearate, CAS number 9004-99-3), tyloxapol, polysorbate (TWEEN), PPG-5-ceteth-20, PEG-20 sorbitan isostearate, alkoxylated alcohols, alkoxylated polyol esters, decyl glucoside, coco-glucoside, sucrose laurate, sorbitol, polyoxyethylene, sorbitan monolaurate and any combination hereof.

According to some embodiments, the non-ionic surfactant is a polyoxyether of lauryl alcohol. According to one embodiment, the polyoxyether of lauryl alcohol is selected from the group consisting of Laureth-1, Laureth-2, Laureth-3, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-21, Laureth-25, Laureth-30, Laureth-38, Laureth-40 and Laureth-50. The number in the name indicates the average number of units of ethylene oxide in the molecule. According to one embodiment, the non-ionic surfactant is selected from Laureth-16, Laureth-20, Laureth-21, Laureth-25, Laureth-30, Laureth-38, and Laureth-40. According to one embodiment, the non-ionic surfactant is Laureth-40. According to some embodiments, the surfactant is Laureth comprising from 20 to 60 units of ethylene oxide. According to other embodiments, the surfactant is Laureth comprising from 30 to 50 units of ethylene oxide. According to some embodiments, the composition comprises from 0.5 to 15 wt % of Laureth having from 20 to 60 units, such as Laureth 40. According to one embodiment, the composition comprises from 1 to 10 wt %, from 2 to 8 or from 3 to 5 wt % of Laureth surfactant, such as Laureth-40. According to some embodiments, the composition comprises from 0.5 to 15 wt % of Disponil 4060 AFX. According to one embodiment, the composition comprises from 1 to 10 wt %, from 2 to 8 or from 3 to 5 wt % of Disponil 4060 AFX. According to some embodiments, the composition comprises from 0.5 to 15 wt % of Sabowax SE40. According to one embodiment, the composition comprises from 1 to 10 wt %, from 2 to 8 or from 3 to 5 wt % of Sabowax SE40.

Nonionic surfactants, can be broadly defined as compounds produced by the condensation of alkylene oxide groups (hydrophilic in nature) with an organic hydrophobic compound, which may be aliphatic or alkyl aromatic in nature. Generally, the surfactant is a non-ionic alkylene oxide condensate of an organic compound which contains one or more hydroxyl groups. For example, ethoxylated and/or propoxylated alcohol or ester compounds or mixtures thereof are commonly available and are well known to those skilled in the art. Suitable surfactants include, but are not limited to, tyloxapol; POLOXAMER 4070; POLOXAMER 188; POLYOXYL 40 Stearate; POLYSORBATE 80, and POLYSORBATE 20, as well as various compounds sold under the trade name TWEEN (ICI American Inc., Wilmington, Del. U.S.A.), PLURONIC F-68 (trade name of BASF, Ludwigshafen, Germany for a copolymer of polyoxyethylene and polyoxypropylene).

According to any one of the above embodiments, the surfactant is a pharmaceutical or cosmetic grade surfactant. The terms “pharmaceutical grade” and “pharmaceutically acceptable” are used herein interchangeably and refer to molecular entities such as surfactants and compositions that do not produce an adverse, allergic, or other untoward reactions when administered to an animal, or human, as appropriate. For human administration, preparations should meet sterility, pyrogenicity, general safety, and purity standards as required by a government drug regulatory agency, e.g., the United States Food and Drug Administration (FDA) Office of Biologics standards or the European medicines agency (EMA). According to some embodiments, the surfactant is a surfactant approved by FDA for human use. The terms “cosmetic grade” and “cosmetically acceptable” are used herein interchangeably and refer to any ingredient, such as surfactant, that is suitable for use in contact with the skin or hair of humans and lower animals without undue toxicity, incompatibility, instability, irritation, allergic response, and the like.

According to some embodiments, any other surfactant that can be applied on the skin and hair for a long period of time, may be used instead of or in addition to non-ionic surfactant. According to some embodiments, the surfactant that may be used instead of or in addition to non-ionic surfactant is a cationic, anionic, amphoteric and zwitterionic surfactant.

Non-limiting examples of cationic surfactants include quaternary ammonium salts, e.g., tetramethylammonium halides, alkyltrimethylammonium halides in which the alkyl group has from about 8 to 22 carbon atoms, for example octyltrimethylammonium chloride, dodecyltrimethylammonium chloride, hexadecyltrimethylammonium chloride, cetyltrimethylammonium chloride, and behenyltrimethylammonium chloride, benzyltrimethylammonium chloride, octyldimethylbenzyl-ammonium chloride, decetyldimethylbenzylammonium chloride, stearyldimethylbenzylammonium chloride, distearyldimethylammonium chloride, didodecyldimethylammonium chloride, dioctadecyldimethylammonium chloride, tallow trimethylammonium chloride, cocotrimethylammonium chloride, cetylpyridinium chloride and the other corresponding halides and hydroxides

Amphoteric and zwitterionic surfactants suitable for use in compositions of the invention may include alkyl amine oxides, alkyl betaines, alkyl amidopropyl betaines, alkyl sulfobetaines, alkyl glycinates, alkyl carboxyglycinates, alkyl amphopropionates, alkulamphoglycinates, alkyl amidopropyl hydroxy-sultaines, acyl taurates and acyl glutamates, wherein the alkyl and acyl groups have from about 8 to 19 carbon atoms. Examples include lauryl amine oxide, cocodimethyl sulphopropyl betaine and preferably lauryl betaine, cocamidopropyl betaine and sodium cocamphoproprionate.

Suitable anionic surfactants are the alkyl sulfonates, alkyl ether sulfonates, alkylaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates, especially their sodium, magnesium, ammonium and mono-, di- and triethanolamine salts. The alkyl and acyl groups generally contain from 8 to 18 carbon atoms and may be unsaturated. The alkyl ether sulfates, alkyl ether phosphates and alkyl ether carboxylates may contain from one to 10 ethylene oxide or propylene oxide units per molecule.

According to other embodiments, the composition further comprises additional surfactants such as sodium dodecyl sulfate or alkyl benzene sulfonates. According to some embodiments, the alkyl benzene sulfonate is selected from branched alkyl benzene sulfonate and linear alkyl benzene sulfonate. According to another embodiment, linear alkyl benzene sulfonate is sodium dodecylbenzene sulfonate. According to one embodiment, the composition comprises sodium dodecylbenzene sulfonate. According to some embodiments, the composition comprises from 0.01 wt % to about 10 wt of sodium dodecylbenzene sulfonate. According to one embodiment, the composition comprises from about 0.01 wt % to about 3 wt % or from 0.05 wt % to about 2 wt % or from 0.1 wt % to about 1.5 wt % of sodium dodecylbenzene sulfonate. According to another embodiment, the composition comprises from about 0.2 wt % to about 1 wt % or from about 0.3 wt % to about 0.9 wt % of sodium dodecylbenzene sulfonate. According to any one of the above embodiments, the surfactant is safe for use in human, and in particular in for use in cosmetics e.g. such as for use in hair care. According to any one of the above embodiments, the surfactant is a pharmaceutical or cosmetic grade surfactant.

According to one embodiment, the composition comprises a combination of polyoxyethers of lauryl alcohol, e.g. Laureth-16, Laureth-20, Laureth-21, Laureth-25, Laureth-30, Laureth-38, or Laureth-40 and linear alkyl benzene sulfonate. According to one embodiment, the composition comprises Laureth-40 and sodium dodecylbenzene sulfonate. According to one embodiment, the composition comprises from about 3 wt % to about 8 wt % Laureth-40 and from about 0.01 wt % to about 1 wt % sodium dodecylbenzene sulfonate.

According to any one of the above embodiments, the amount of solids in said composition is from about 15 wt % to about 55 wt %. According to one embodiment, the amount of solids is from about 20 to about 45 wt %. According to another embodiment, the amount of solids is from about 20 to about 40 wt %. According to yet another embodiment, the amount of solids in said composition is from about 22 wt % to about 38 wt % or from about 24 wt % to about 36 wt %.

According to any one of the above embodiments, the glass transition temperature (Tg) of the polymer is from about 10° C. to about 60° C. According to some embodiments, the Tg is from about 10° C. to about 50° C. or to about 40° C. According to yet another embodiment, the Tg is from about 15° C. to about 35° C.

According to any one of the above embodiment, the polymers undergo crosslinking upon drying. The crosslinking occurs via the cross-linking unit of the polymer. According to one embodiment, the crosslinking is a complete crosslinking. According to another embodiment, the crosslinking is a partial crosslinking. Partial crosslinking means that only a part of cross-linkers reacted to form a bond with another polymer. According to some embodiments, partial crosslinking encompasses that about 10 to 90% of the cross-linkers reacted. According to another embodiment, partial crosslinking encompasses that about 20% to about 80%, about 30% to about 70%, or about 40% to about 60% of the cross-linkers bound to another polymer. According to some embodiments, upon crosslinking the polymers form a film around hair.

According to any one of the above embodiments, the straightening composition further comprises a preservative. According to one embodiment, the preservative is selected from propylene glycol, methyl propanediol, caprylyl glycol, polyquaternium-80, didecyldimium chloride, ethylhexylglycerin, Dehydroacetic acid (2-Acetyl-5-hydroxy-3-oxo-4-hexenoic acid 6-lactone), paraben, methyl paraben, phenoxyethanol, iodopropynyl butyl carbamate, and diazolidinyl urea.

According to some embodiments, the present invention provides a hair straightening composition comprising from about 5 wt % to about 45 wt % of at least one acrylic-based polymer comprising an acrylic-based cross-linker wherein the amount of said cross-linker is from about 2.5 wt % to about 25 wt % of said acrylic-based polymer, and from about 0.5 wt % to about 10 wt % of a non-ionic surfactant, wherein the non-ionic surfactant is selected from a pharmaceutical or cosmetic grade surfactant, wherein the composition has Tg from about 10° C. to about 50° C. and wherein the composition straightens the hair or preserves hair in straightened conformation upon drying. According to such embodiments, the cross-linker is AAEM. According to some such embodiments, the polymer is derived from a combination of acrylic monomers and acrylic oligomers, the combination is selected from the group consisting of (i) monoacrylate oligomer and isobornyl acrylate; (ii) monoacrylate oligomer, isobornyl acrylate and butyl acrylate; and (iii) monoacrylate oligomer, isobornyl acrylate and lauryl methacrylate. According to some such embodiments, the polymers undergo crosslinking upon drying. According to such embodiments, the non-ionic surfactant is selected from Laureth-20, Laureth-21, Laureth-25, Laureth-30, Laureth-38, Laureth-40, Disponil 4060 AFX, and Sabowax SE40. According to some embodiments, the hair straightening composition comprising from about 15 wt % to about 40 wt % of at least one acrylic-based polymer comprising an acrylic-based cross-linker and from about 2 wt % to about 8 wt % of a non-ionic surfactant, wherein the amount of said cross-linker is from about 2.5 wt % to about 25 wt % of said acrylic-based polymer and the non-ionic surfactant is selected from a pharmaceutical or cosmetic grade surfactant, the composition has Tg of from 10 to 45° C., the polymers undergo crosslinking upon drying and the composition straightens the hair upon drying. According to some embodiments, such composition comprises sodium dodecylbenzene sulphonate. According to one embodiment, the composition comprises from about 3 wt % to about 8 wt % Laureth having from 20 to 40 units of ethylene oxide and from about 0.2 wt % to about 1 wt % sodium dodecylbenzene sulfonate. According to such embodiments, the polymer comprises from 85 to 100% of acrylic moieties or consists of acrylic moieties.

According to some embodiment the present invention provides a hair straightening composition comprising from about 15 wt % to about 35 wt % of at least one acrylic-based polymer comprising an acrylic-based cross-linker and from about 2 wt % to about 8 wt % of a non-ionic surfactant, wherein the amount of said cross-linker is from about 5 wt % to about 20 wt % of said acrylic-based polymer and the non-ionic surfactant is selected from a pharmaceutical or cosmetic grade surfactant, wherein the composition has Tg from about 10° C. to about 50° C. and wherein the composition straightens the hair or preserves hair in straightened conformation upon drying. According to such embodiments, the cross-linker is AAEM and the polymer is derived from a combination of acrylic monomers and acrylic oligomers, the combination is selected from the group consisting of (i) a combination of monoacrylate oligomer and isobornyl acrylate; (ii) a combination monoacrylate oligomer, isobornyl acrylate and butyl acrylate; and (iii) a combination monoacrylate oligomer, isobornyl acrylate and lauryl methacrylate, and the non-ionic surfactant is Laureth having from 20 to 40 units of ethylene oxide such as Laureth-20, Laureth-21, Laureth-25, Laureth-30, Laureth-38, Laureth-40, Disponil AFX 4060, and Sabowax SE40. According to some such embodiments, the polymers undergo crosslinking upon drying. According to one embodiment, the monoacrylate oligomer is 2-hydroxy-3-(prop-2-enoyloxy)propyl 2-methyl-2-propylhexanoate. According to some embodiments, such a composition further comprises from about 0.1 wt % to about 1 wt % of sodium dodecylbenzene sulphonate. According to such embodiments, the polymer comprises from 95 to 100% of acrylic moieties or consists of acrylic moieties.

According to one embodiment, the hair straightening composition comprises (i) an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, AAEM in the amount of from 8 to about 20 wt % of the polymer; (ii) from about 3 to about 6 wt % of Laureth-40, Disponil AFX 4060, or Sabowax SE40 and (iii) from 0.2 to about 1 wt % of sodium dodecylbenzene sulphonate. According to another embodiment, the polymer further comprises lauryl methacrylate units in the amount of from about 1 to about 5 wt % of the polymer.

The term comprising has also the meaning of consisting or consisting essentially of. Thus, in one embodiment, the hair straightening composition consists essentially of (i) an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, AAEM in the amount of from 8 to about 20 wt % of the polymer, lauryl methacrylate units in the amount of from about 1 to about 5 wt % of the polymer; (ii) from about 3 to about 6 wt % of Laureth-40, Disponil AFX 4060, or Sabowax SE40 and (iii) from 0.2 to about 1 wt % of sodium dodecylbenzene sulphonate. According to another embodiment, the hair straightening composition consists essentially of (i) an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, AAEM in the amount of from 8 to about 20 wt % of the polymer; (ii) from about 3 to about 6 wt % of Laureth-40, Disponil AFX 4060, or Sabowax SE40 and (iii) from 0.2 to about 1 wt % of sodium dodecylbenzene sulphonate.

According to any one of the above embodiments, the obtained film acts as a surface barrier and/or moisture shield around hair.

According to some embodiments, the content of the initial composition used for preparation of the composition of the present invention is as described in Table 1 and in Example 4. Each composition in these tables represent a separate embodiment. According to some particular embodiments, the composition comprises from 25 to 50 wt % of polymer obtained by polymerization of from 6 to 14 wt % of monoacrylate oligomer CN 152, from 8 to 15 wt % of isobornyl acrylate, optionally from 1 to 5 wt % of lauryl methacrylate, from 2 to 5 wt % of AAEM in the presence of from 3 to 8 wt % Laureth-40 and from 0.2 to 1 wt % of sodium dodecylbenzene sulphonate. The polymerization reaction is initiated by ammonium persulphate, e.g 0.05 wt %.

According to some embodiments, the polymer of the present invention is devoid of maleic anhydride moieties. According to some embodiments, the polymer of the present invention is devoid of maleimide moieties. According to one embodiment, the polymer comprises less than 5 wt %, less than 3 wt % or less than 1 wt % of said moieties. The terms “substantially devoid”, “essentially devoid”, “devoid”, “does not include” and “does not comprise” may be used interchangeably and refer to composition that does not include, contain or comprise a particular component, e.g. said composition comprises less than 0.1 wt %, less than 0.01 wt %, or less than 0.001 wt % of the component. In some embodiments, the term devoid contemplates composition comprising traces of the devoid component such as traces of a component used in purification process.

According to another aspect, the present invention provides a hair care formulation comprising from about 3 wt % to 40 wt % of the hair styling composition of the present invention. As such, the hair care formulation of the present invention is a hair styling formulation. According to one embodiment, the hair styling composition is a hair straightening composition. Thus, in some embodiments, the hair straightening formulation comprises from about 3 wt % to about 40 wt % of the hair straightening composition of the present invention.

As used here, the term “formulation” refers to the final hair care cosmetic product. According to some embodiments, the formulation is a cosmetic formulation. According to other embodiments, the formulation is ready to use hair care formulation, which may be used directly on hair.

According to one embodiment, the hair care formulation, i.e. the hair straightening formulation comprises from about 2 wt % to about 40 wt % of a hair straightening composition, wherein the hair straightening composition comprises from about 5 wt % to about 55 wt % of at least one acrylic-based polymer comprising acrylic units that act as cross-linkers wherein the amount of the cross-linker is from about 2.5% to about 25 wt % of said acrylic-based polymer, and from about 0.5 wt % to about 15 wt % of a non-ionic surfactant, wherein the non-ionic surfactant is selected from a pharmaceutical or cosmetic grade surfactant, and wherein the polymers of the composition undergo cross linking and thereby straighten the hair upon drying. According to some embodiments, the formulation comprises a cosmetically acceptable carrier. According to one embodiment, the formulation preserves hair in straightened conformation. According to one embodiment, the cross-linkers form bonds between the polymer chains upon drying of the formulation. All embodiments, definitions and limitations defined for a hair straightening composition in previous aspects are encompassed herein.

According to one embodiment, the hair straightening formulation comprises from about 3 wt % to about 40 wt % of a hair straightening composition comprising from about 5 wt % to about 45 wt % of at least one acrylic-based polymer comprising an acrylic-based cross-linker and from about 0.5 wt % to about 10 wt % of a non-ionic surfactant, wherein the amount of said cross-linker is from about 2.5 wt % to about 25 wt % of said acrylic-based polymer and the non-ionic surfactant is selected from a pharmaceutical or cosmetic grade surfactant, wherein the formulation has Tg from about 10° C. to about 50° C. and wherein the formulation straightens the hair upon drying. According to such embodiments, the cross-linker is AAEM. According to some such embodiments, the polymer is derived from a combination of acrylic monomers and acrylic oligomers, the combination is selected from the group consisting of (i) monoacrylate oligomer and isobornyl acrylate; (ii) monoacrylate oligomer, isobornyl acrylate and butyl acrylate; and (iii) monoacrylate oligomer, isobornyl acrylate and lauryl methacrylate. According to some such embodiments, the polymer comprises from 85 to 100 wt % of acrylic moieties. According to some such embodiments, the polymer undergoes crosslinking upon drying of the formulation. According to some embodiments, the hair straightening composition comprising from about 15 wt % to about 40 wt % of at least one acrylic-based polymer comprising an acrylic-based cross-linker and from about 2 wt % to about 8 wt % of a non-ionic surfactant, wherein the amount of said cross-linker is from about 2.5 wt % to about 25 wt % of said acrylic-based polymer and the non-ionic surfactant is selected from a pharmaceutical or cosmetic grade surfactant, the formulation has Tg of from 10 to 45° C., the polymers undergo crosslinking upon drying of the formulation, and the formulation straightens the hair upon drying. According to one embodiment, the monoacrylate oligomer is 2-hydroxy-3-(prop-2-enoyloxy)propyl 2-methyl-2-propylhexanoate.

According to one embodiment, the hair straightening formulation comprises from about 3 wt % to about 20 wt % of a hair straightening composition comprising from about 15 wt % to about 35 wt % of at least one acrylic-based polymer comprising an acrylic-based cross-linker and from about 2 wt % to about 8 wt % of a non-ionic surfactant, wherein the amount of said cross-linker is from about 5 wt % to about 20 wt % of said acrylic-based polymer and the non-ionic surfactant is selected from a pharmaceutical or cosmetic grade surfactant, wherein the formulation has Tg from about 10° C. to about 50° C. and wherein the formulation straightens the hair or preserves hair in straightened conformation upon drying. According to such embodiments, the cross-linker is AAEM, the polymer is derived from a combination of acrylic monomers and oligomers, the combination selected from the group consisting of (i) a monoacrylate oligomer and isobornyl acrylate; (ii) a monoacrylate oligomer, isobornyl acrylate and butyl acrylate; and (iii) a monoacrylate oligomer, isobornyl acrylate and lauryl methacrylate, and the non-ionic surfactant is laureth having from 20 to 40 units of ethylene oxide such as Laureth-20, Laureth-21, Laureth-25, Laureth-30, Laureth-38, Laureth-40, Disponil AFX 4060 and Sabowax SE40. According to some such embodiments, the polymers undergo crosslinking upon drying. According to some embodiments, such composition further comprises from about 0.1 wt % to about 1 wt % of sodium dodecylbenzene sulphonate. According to some such embodiments, the polymer comprises from 85 to 100% of acrylic moieties. According to one embodiment, the monoacrylate oligomer is 2-hydroxy-3-(prop-2-enoyloxy)propyl 2-methyl-2-propylhexanoate.

According to any one of the above embodiments, the hair straightening formulation comprises from about 3 wt % to about 35 wt % of the hair straightening composition of the present invention. According to one embodiment, the hair straightening formulation comprises from about 5 wt % to about 30 wt % of the hair straightening composition of the present invention. According to one embodiment, the hair straightening formulation comprises from 4 wt % to about 25 wt % of the hair straightening composition of the present invention. According to other embodiment, the hair straightening formulation comprises from 10 wt % to about 20 wt %, or 5 wt %, 10 wt %, 15 wt %, 20 wt % or 25 wt %% of the hair straightening composition according to any one of the above embodiments. According to another embodiment, the formulation comprises from 2 wt % to 20 wt % of the hair straightening composition of the present invention. According to a further embodiment, the formulation comprises from about 3 wt % to about 18 wt % or from about 4 wt % to about 15 wt % of the hair straightening composition of the present invention. According to some embodiments, the hair straightening formulation comprises from about 2 wt % to about 25 wt % of the hair straightening composition. According to another embodiment, the hair straightening formulation comprises from about 3 wt % to about 20 wt %, from 4 to 15 wt % or from 5 to 10 wt % of the hair straightening composition. According to one embodiment, the hair straightening formulation comprises from about 3 wt % to about 15 wt % of the hair straightening composition.

According to any one of the above embodiments, the length of the straightened hair upon drying the formulation is up to 200%, up to 180%, up to 160%, or up to 150%, of the hair's length in un-straightened conformation. According to one embodiment, the length of the straightened hair upon drying the formulation is up to 140%, of the hair's length in un-straightened conformation. According to another embodiment, the length of the straightened hair upon drying the formulation is from 101% to 130%, 105% to 125%, or from 110% to 120% of the hair's length in un-straightened conformation. The un-straightened conformation means the conformation of dry and untreated hair.

According to some embodiments, the formulation of the present invention preserves or maintains hair in the straightened conformation from several hours to several days. According to one embodiment, the formulation of the present invention preserves or maintains hair in the straightened conformation from 1 to 24, from 3 to 20 from 4 to 16 from 6 to 12 hours, provided that that hair is not wetted, washed or shampooed. According to one embodiment, the formulation of the present invention preserves hair in the straightened conformation for from 1 to 5, from 2 to 4, or for 3 days provided that that hair is not wetted, washed or shampooed. According to other embodiments, the formulation of the present invention preserves hair in the straightened conformation for 3 to 14, for 5 to 12 or for 7 to 10 days.

According to some embodiments, the formulation of the present invention preserves hair in the straightened conformation under high humidity conditions. According to one embodiments, the formulation of the present invention preserves hair in the straightened conformation for 1, 2, 4 or 5 days in relative humidity (RH) of more than 50%, 60%, 70% or 80%. The term “relative humidity” as used herein refers to the amount of water vapor present in air expressed as a percentage of the amount needed for saturation at the same temperature (i.e. relative to the “dew point”).

According to any one of the above embodiments, the hair care formulation, e.g. the hair straightening formulation of the present invention is selected from the group consisting of a spray, lotion, cream, foam, mousse, and gel.

According to any one of the above embodiments, the hair care formulation, e.g. the hair straightening formulation of the present invention further comprises one of more additional ingredients. According to one embodiment, the ingredients are cosmetically acceptable ingredients. According to some embodiments, the additional ingredients are selected from the group consisting of preservatives, thickeners, viscosity modifiers, fatty alcohols, pH adjusting agents, buffers, coloring agents, guar gums, natural gums, perfumes, PEGs, essential oils, silicone fluid dimethicone, hydroxypropyl cellulose, carboxymethyl cellulose and any combination thereof.

The pH adjusting agents can be selected from, but are not limited to, glyoxylic acid, maleic acids, succinic acid, formic acid and other organic and inorganic acids.

According to one embodiment, the additional ingredient is a preservative or a combination of preservatives. propylene glycol, paraben, methyl paraben, phenoxyethanol, caprylyl glycol, iodopropynyl butyl carbamate, and diazolidinyl urea.

According to one embodiment, the essential oil is selected from coconut, lime and argan oil. According to another embodiment, the essential oil is selected from lavender, rosemary, chamomile, cedar-wood, clary sage, and peppermint oil.

The hair care formulations described hereinabove can also contain a variety of other optional cosmetically acceptable components suitable for rendering such formulations more acceptable. Such conventional optional ingredients are well known to those skilled in the art, e.g., pearlescent aids such as ethylene glycol distearate; preservatives such as benzyl alcohol, methyl paraben, propyl paraben and imidazolidinyl urea; thickeners and viscosity modifiers such as a diethanolamide of a long chain fatty acid (e.g., PEG 3 lauric diethanolamide), cocomonoethanol amide, dimethicone copolyols, guar gum, methyl cellulose starches and starch derivatives, fatty alcohols such as stearyl alcohol, sodium chloride, sodium sulfate, polyvinyl alcohol, and ethyl alcohol; pH adjusting agents such as citric acid, sodium citrate, succinic acid, phosphoric acid, sodium hydroxide, sodium carbonate, etc.; propellants; surfactants; coloring agents such as any of the FD&C or D&C dyes hair oxidizing (bleaching) agents such as hydrogen peroxide, perborate and persulfate salts, hair reducing agents such as the thioglycolates; perfumes; and, sequestering agents such as disodium ethylenediamine tetraacetate, polymer plasticizing agents such as glycerin and propylene glycol. According to some embodiments, said agents generally used individually or collectively at a level of from about 0.01 wt % to about 20 wt %, or from about 0.05 wt % to about 18 wt %, from about 1 wt % to about 10 wt %, from about 2 wt % to about 8 wt % or from about 3 wt % to about 5.0 wt %.

According to one embodiment, the hair straightening formulation comprises at least one of the excipients selected from Sharomix Preservative, BEG (ethyl hexyl glycerin, benzyl alcohol, d-alpha-tocopherol 10:89.8:0,2), CP10 (phenoxyethanol:chlorophenesin 90:10), Gum guar, perfume, PEG-12 dimethicone, Silicone fluid Dimethicone (Q7-9120 or 12500 CT), Polydimethylsiloxane (PDMS-350CST), Hydroxypropyl cellulose (Klucel), Carboxymethylcellulose sodium, and Argan oil, as described in Table 2. According to some embodiments, the hair straightening formulation is a formulation as described in Table 3. Each one of said formulations represents a separate embodiment.

According to some embodiments, the straightening formulation comprises from about 3 to about 15 wt of a hair straightening composition, wherein the hair straightening composition comprises (i) an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, and AAEM in the amount of from 8 to about 20 wt % of the polymer; (ii) from about 3 to about 6 wt % of Laureth-40, Disponil AFX 4060 or Sabowax SE40 and (iii) from 0.2 to about 1 wt % of sodium dodecylbenzene sulphonate. According to another embodiment, the polymer further comprises lauryl (meth)acrylate units in the amount of from about 1 to about 5 wt % of the polymer. According to some embodiments, the formulation further comprises at least one of the compositions selected from Sharomix Preservative, BEG (ethyl hexyl glycerin, benzyl alcohol, d-alpha-tocopherol 10:89.8:0,2), CP10 (phenoxyethanol:chlorophenesin 90:10), Gum guar, perfume, PEG-12 dimethicone, Silicone fluid Dimethicone (Q7-9120 or 12500 CT), polydimethylsiloxane, hydroxypropyl cellulose, Carboxymethylcellulose sodium, Argan oil and a combination thereof. According to some embodiments, each one of said components may be in the amount of 0.1 to 30 wt % of the formulation. According to one embodiment, the straightening formulation comprises from about 3 to about 15 wt of a hair straightening composition, wherein the hair straightening composition consists essentially of (i) an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, AAEM in the amount of from 8 to about 20 wt % of the polymer, lauryl methacrylate units in the amount of from about 1 to about 5 wt % of the polymer; (ii) from about 3 to about 6 wt % of Laureth-40, Disponil AFX 4060, or Sabowax SE40 and (iii) from 0.2 to about 1 wt % of sodium dodecylbenzene sulphonate. According to another embodiment, the hair straightening composition consists essentially of (i) an acrylic based polymer comprising 2-hydroxy-3-(prop-2-enoyloxy)propyl 2-methyl-2-propylhexanoate units in the amount of 35 to 55 wt % of the polymer, isobornyl acrylate units in the amount of 40 to 60 wt % of the polymer, AAEM in the amount of from 8 to about 20 wt % of the polymer; (ii) from about 3 to about 6 wt % of Laureth-40, Disponil AFX 4060, or Sabowax SE40 and (iii) from 0.2 to about 1 wt % of sodium dodecylbenzene sulphonate.

According to any one of the above embodiments, the formulation comprises from about 50 wt % to about 90 wt % of carrier. According to some embodiment, the formulation comprises from 55 to 85 wt % of from 60 to 80 wt % carrier. According to one embodiment, the carrier is water.

According to a further aspect, the present invention provides a method of preparation of the hair straightening formulation of the present invention comprising dissolving the hair straightening composition of the present invention in cosmetically acceptable carrier such as water and optionally adding one or more cosmetically acceptable excipient to obtain a formulation comprising from about 3 wt % to about 40 wt % of the hair straightening composition.

According to yet another aspect, the present invention provides a method of hair styling comprising applying the hair styling formulation or the hair styling composition of the present invention to the hair and drying the formulation or composition, respectively. According to one embodiment, the hair styling is hair straightening. Thus, in one embodiment, the present invention provides a method of hair straightening comprising applying the hair straightening formulation to the hair and drying the formulation. According to another embodiment, the present invention provides a method of hair straightening comprising applying the hair straightening composition to the hair and drying the composition. According to yet another embodiment, the present invention provides a method of preserving hair in a straightened conformation, e.g. after wetting the hair, the method comprises applying hair straightening composition or formulation of the present invention to the hair and drying the composition or formulation, respectively. As used herein, the term “applying” a formulation to the hair or “treating” the hair with a formulation is intended to mean contacting the hair with at least one of the formulations of the invention, in any manner.

According to one embodiment, the drying is drying by heat, e.g. by a fan. According to another embodiment, the drying is by drying at room temperature.

According to any one of the above embodiments, the method comprises combing or brushing the hair to straighten it before drying.

According to any one of the above embodiments, the method comprises leaving the formulation for about 1 to about 10 min on hair before drying. According to some embodiments, the formulation is left for about 2 to about 8 of for about 3 to about 6 minutes.

According to any one of the above embodiment, applying the formulation comprises applying the formulation on wet hair. According to another embodiment, applying of the formulation is applying to dry hair.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The term “or,” as used herein, denotes alternatives that may, where appropriate, be combined; that is, the term “or” includes each listed alternative separately as well as their combination if the combination is not mutually exclusive.

Having now generally described the invention, the same will be more readily understood through reference to the following examples, which are provided by way of illustration and are not intended to be limiting of the present invention.

### EXAMPLES

The following non-limiting examples describe the synthesis of self-cross-linking acrylic emulsions and their incorporation into personal care, hair-straightening formulations.

### Example 1: Synthesis of an Emulsion Comprising Acrylic Polymers with Crosslinking Units

A general procedure for preparation of the acrylic copolymers comprising crosslinking units is described.

Pre-Emulsion Preparation:

37.5 g sodium dodecylbenzene sulfonate was completely dissolved in 242 g deionized water by mixing with a magnetic stirrer. Then 27 g Laureth-40 (60% in deionized water) were added to form a transparent viscous surfactant solution. In a 0.5 liter beaker were placed all of the monomers (see below). This monomer solution was poured into the surfactant solution and mixed to give a pre-emulsion. The pre-emulsion was kept for 20 hours in a refrigerator.

Monomers/Oligomers:

60 g CN152 (Sartomer), Epoxy acrylate oligomer

67.5 g isobornyl monoacrylate,

22.5 g 2-(methacryloyloxy)ethylacetoacetate (AAEM)—the cross-linker,

Water, 342 g.

CN152 of Sartomer comprises 2-hydroxy-3-(prop-2-enoyloxy)propyl 2-methyl-2-propylhexanoate as a main component. It may contain traces of the initial ingredients such as 2,3-Epoxypropyl neodecanoate, neodecanoic acid 1-ester, 1,2,3-propanetriol mono-2-propenoate, acrylic acid and mequinol.

Polymerization

To the pre-emulsion 300 mg ammonium persulfate were added and it was stirred with a magnetic stirrer for 0.5 h. 100 ml of water were added to the reactor and the mechanical stirrer was set at 300 rpm. Then 100 ml of pre-emulsion were added. Nitrogen gas was purged through the mixture for 30 minutes. Some foam may be formed at this stage. The reactor was then placed in an oil bath and heated to 84° C. and then the remainder of the pre-emulsion was added to the reactor during two hours and 10 minutes using a peristaltic pump. To complete the polymerization, 30 mg of ammonium per sulfate were added and the temperature was raised to 95-97° C. for an additional hour. The heating was shut off and the contents of the reactor were cooled slowly with continuous stirring with the reactor remaining in the oil bath. The emulsion polymer product, a synthetic latex, was removed from the reactor and filtered through a synthetic non-woven cloth. No coagulation product was observed. The pH of the emulsion was 2.7. The pH was raised to 6.8 by carefully adding about twenty drops of 25% ammonium hydroxide. This was monitored using a pH electrode. The final yield was 500 ml.

Analyses

The resulting polymer emulsion (composition) was analyzed for its glass transition temperature, Tg, and the solid content. The Tg by Differential Scanning calorimeter (DSC) was 17° C., and the total solids content was 32.8%.

Several additional polymers with different content of the monomers/oligomers were prepared as described above. Table 1 summarizes the initial ingredients and properties of the obtained products.

TABLE 1
Different preparations of compositions.

| | 1140 | | batch 2-18-2 | | batch 2-18-7 | | batch 2-18-15 | | batch 2-18-19 | | batch 2-18-24 | |
|---|---|---|---|---|---|---|---|---|---|---|---|---|
| Materials | wt, g | wt % | wt, g | wt % | wt, g | wt % | wt, g | wt % | wt, g | wt % | wt, g | wt % |
| Monoacrylate | 60 | 11.54 | 60 | 9.63 | 60 | 9.81 | 60 | 11.47 | 60 | 11.47 | 60 | 11.4 |
| Oligomer CN | | | | | | | | | | | | |
| 152 | | | | | | | | | | | | |
| Isobornyl | 67.5 | 12.9 | 67.5 | 10.8 | 67.5 | 11.0 | 54 | 10.3 | 54 | 10.32 | 67.5 | 12.8 |
| Acrylate (Sigma) | | | | | | | | | | | | |
| Butyl Acrylate | | | | | | | 13.5 | 2.58 | — | | — | — |
| Lauryl | | | | | | | | | 13.5 | 2.58 | — | — |
| Methacrylate | | | | | | | | | | | | |
| AAEM | 22.5 | 4.30 | 22.5 | 3.61 | 11 | 1.80 | 22.5 | 4.3 | 22.5 | 4.30 | 27 | 5.1 |
| (cross-linker) | | | | | | | | | | | | |
| Laureth-40 | 27 | 5.16 | 27 | 4.33 | 27 | 4.41 | 27 | 5.2 | 27 | 5.16 | 27 | 5.1 |
| (non-ionic | | | | | | | | | | | | |
| surfactant) | | | | | | | | | | | | |
| Sodium | 3.75 | 0.72 | 3.75 | 0.60 | 3.75 | 0.61 | 3.75 | 0.72 | 3.75 | 0.72 | 3.75 | 0.71 |
| Dodecylbenzene | | | | | | | | | | | | |
| sulphonate | | | | | | | | | | | | |
| (surfactant) | | | | | | | | | | | | |
| Ammonium | 0.33 | 0.06 | 0.33 | 0.05 | 0.33 | 0.05 | 0.33 | 0.06 | 0.33 | 0.06 | 0.33 | 0.06 |
| persulphate | | | | | | | | | | | | |
| DIW | 342 | 65.48 | 442 | 70.9 | 442 | 72.27 | 342 | 65.38 | 317 | 65.38 | 342 | 64.82 |
| Total | 523. | 100. | 623.0 | 100.0 | 611.5 | 100.0 | 523.0 | 100.0 | 527.5 | 100.0 | 527.5 | 100.0 |
| Percent of | 32.5 | | | 27.3 | | 26.0 | | 32.5 | | 34.1 | | 33.1 |
| solids | | | | | | | | | | | | |
| calculated, % | | | | | | | | | | | | |
| % solids by | | | | 26.7 | | | | 34.6 | | — | | — |
| lyophilization | | | | | | | | | | | | |
| Percent | | | | 26.6 | | 26.1 | | 34.9 | | 35.4 | | 33.8 |
| solids by | | | | | | | | | | | | |
| DSC, % | | | | | | | | | | | | |
| Tg ° C. | 17 | | | | | 44.1 | | 38.4 | | NA | | N.A. |
| by DSC | | | | | | | | | | | | |
| Ammonium | 12 | | 12 | | 12 | | 10 | | 12 | | 18 | |
| Hydroxide, 25% | | | | | | | | | | | | |
| (drops) | | | | | | | | | | | | |

### Example 2: Preparation of Hair Straightening Formulation

Table 2 lists chemicals and auxiliary materials that may be used in the hair straightening formulation of the present application.

TABLE 2

| | | Trade | |
|---|---|---|---|
| | Ingredient | Name/Supplier | CAS |
| 1 | Double Deionized Water | Zalion | |
| 2 | Sharomix Preservative | Sharon Labs | BEG: 70445-33-9; |
| | BEG (ethyl hexyl | | 100-51-6; |
| | glycerin, benzyl alcohol, | | 59-02-9 |
| | d-alpha - tocopherol | | CP-10: 122-99-6; |
| | 10:89.8:0.2) or | | 104-29-0 |
| | CP10(phenoxyethanol:chlorophenesin | | |
| | 90:10) | | |
| 3 | Gum guar | Solvay | |
| 4 | Perfume | | |
| 5 | PEG-12 dimethicone/ | Dow Corning | |
| | 193 C Fluid | | |
| 6 | Silicone fluid | Dow corning | 63148-62-9 |
| | Dimethicone | | |
| | Q7-9120 12500 | | |
| | CT or 350 CST - | | |
| | Polydimethylsiloxane | | |
| | (PDMS) | | |
| 7 | Hydroxypropyl | Ashland | EF Ref - |
| | cellulose | | 420705 |
| | Klucel | | HF Ref - |
| | | | 424941 |
| 8 | Carboxymethylcellulose | Spectrum | 9004-32-4 |
| | sodium - CA194 | | |
| 9 | Argan oil/Argania | | |
| | Spinosa | | |

The following hair straightening formulations were prepared by mixing the compositions prepared as described in Example 1 with additional excipients in a homogenizer. The formulations were observed for 24 hours to verify that no phase separation occurs. The content of the hair-straightening composition in the formulation is indicated as “% of stock composition”.

TABLE 3
Ready to use hair straightening formulations comprising the composition of the present invention.

| | Formulation No. | | | | | | | | |
|---|---|---|---|---|---|---|---|---|---|
| | 62-B | 62-2 | 62-19 | 63-2 | 63-19 | 64-2 | 64-19 | 65-2 | 65-19 |
| | Composition Batch | | | | | | | | |
| | 2-18-2 | 2-18-2 | 2-18-19 | 2-18-2 | 2-18-19 | 2-18-2 | 2-18-19 | 2-18-2 | 2-18-19 |
| Ingredient | wt % | wt % | wt % | wt % | wt % | wt % | % | % | wt % |
| Water | 88.4 | 68.4 | 68.4 | 73.4 | 73.4 | 78.4 | 78.4 | 83.4 | 83.4 |
| % of stock | 0 | 20 | 20 | 15 | 15 | 10 | 10 | 5 | 5 |
| composition | | | | | | | | | |
| Guar gum 162 | 0.1 | 0.1 | 0.1 | 0.1 | 0.1 | 0.1 | 0.1 | 0.1 | 0.1 |
| Sharomix BEG | 0.6 | 0.6 | 0.6 | 0.6 | 0.6 | 0.6 | 0.6 | 0.6 | 0.6 |
| PEG-12 dimethicone | 0.2 | 0.2 | 0.2 | 0.2 | 0.2 | 0.2 | 0.2 | 0.2 | 0.2 |
| dimethicone 350 | 0.1 | 0.1 | 0.1 | 0.1 | 0.1 | 0.1 | 0.1 | 0.1 | 0.1 |
| Squalane | 0.1 | 0.1 | 0.1 | 0.1 | 0.1 | 0.1 | 0.1 | 0.1 | 0.1 |
| Argan oil. | 0.2 | 0.2 | 0.2 | 0.2 | 0.2 | 0.2 | 0.2 | 0.2 | 0.2 |
| D-pantanol | 0.2 | 0.2 | 0.2 | 0.2 | 0.2 | 0.2 | 0.2 | 0.2 | 0.2 |
| Perfume | 0.05 | 0.05 | 0.05 | 0.05 | 0.05 | 0.05 | 0.05 | 0.05 | 0.05 |
| Carboxymethyl | 20 | 20 | 20 | 20 | 20 | 20 | 20 | 20 | 20 |
| cellulose - 1% | | | | | | | | | |
| Klucel HF | 0.25 | 0.25 | 0.25 | 0.25 | 0.25 | 0.25 | 0.25 | 0.25 | 0.25 |
| Total solids | 21.8 | 41.8 | 41.8 | 36.8 | 36.8 | 31.8 | 31.8 | 26.8 | 26.8 |

All formulations were stable and no phase separation was observed after 24 hours.

### Example 3. Applying Hair-Straightening Formulation to Hair

The styling cream may be applied on both dry and wet hair. Described herein is a general procedure for applying the hair straightening formulation after washing the hair.

After washing the hair rinse out the shampoo using warm water and wring out the hair removing as much water as possible. Pour a small portion of the formulation into the palm of your hand; about 0.15 g for 1 g of hair, length 30 cm. Run the formulation through the length of the hair, trying to apply it to every strand. Allow the formulation to set for about 1 to 5 min. The longer the time the formulation stays on the hair, the more effective it is in straightening and protecting the hair. Use a wide-tooth comb to comb the hair after applying the leave-in formulation. This will spread the product out even further, and help to avoid clumps of the formulation from making certain sections of the hair greasy while others are left dry.

Start air drying the hair with a fan on a medium heat and high speed setting.

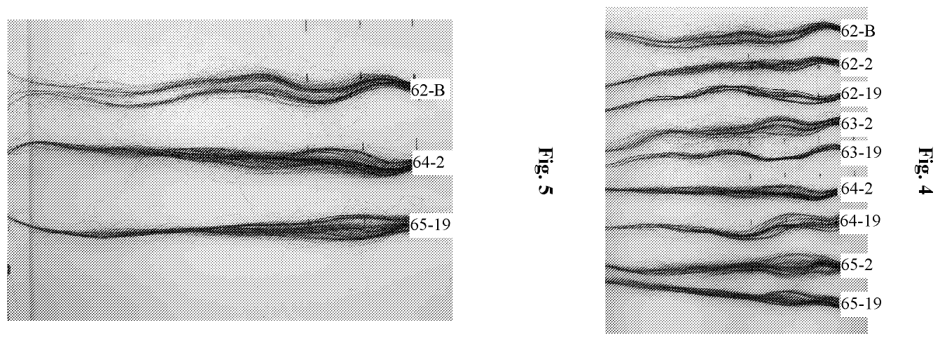

The formulation was applied to Brazilian hair type 2 as described above and the results are shown in FIGS. 3-5. FIG. 3 shows the hair after application of 1 g. of Formulations 64-2, 64-19, 65-2 and 65-19 (see Table 3) to hair. FIG. 4 shows the hair after drying. It can be seen that formulation 64-2 (fourth from right) and 65-19 (extreme right) provided the best results on Brazilian hair type 2. FIG. 5 shows the comparison between hair swatches, treated with a formulation either comprising or not the composition of the present invention. The difference is most clearly seen on the lower halves of the strands.

### Example 4. Preparation of Two Hair-Straightening Compositions

Composition 101

Pre-Emulsion Preparation:

7.5 g sodium dodecylbenzene sulfonate was completely dissolved in 400 g deionized water by mixing with a magnetic stirrer. Then 46.6 g Disponil AFX 4060 (Cognis) (fatty acid alcohol polyglycol ether) were added to form a transparent viscous surfactant solution. In a 1 liter beaker were placed all of the monomers (see below). This monomer solution was poured into the surfactant solution and mixed to give a pre-emulsion. The pre-emulsion was kept for 20 hours in a refrigerator.

Monomers/Oligomers:

110 g CN152 (Sartomer), (2-hydroxy-3-(prop-2-enoyloxy)propyl 2-methyl-2-propylhexanoate)

129 g isobornyl monoacrylate,

41.5 g 2-(methacryloyloxy)ethylacetoacetate (AAEM)—the cross-linker,

Water, 700 g.

Polymerization

To the pre-emulsion 580 mg ammonium persulfate were added and it was stirred with a mechanical stirrer for 0.5 h. 200 ml of water were added to the reactor and the mechanical stirrer was set at 300 rpm. Then 200 ml of pre-emulsion were added. Nitrogen gas was purged through the mixture for 30 minutes. Some foam may be formed at this stage. The reactor was then placed in an oil bath and heated to 84° C. and then the remainder of the pre-emulsion was added to the reactor during two hours and 10 minutes using a peristaltic pump. To complete the polymerization, 58 mg of ammonium per sulfate were added and the temperature was raised to 95-97° C. for an additional hour. The heating was shut off and the contents of the reactor were cooled slowly with continuous stirring with the reactor remaining in the oil bath. The emulsion polymer product, a synthetic latex, was removed from the reactor and filtered through a synthetic non-woven cloth. No coagulation product was observed. The pH of the emulsion was 2.7. The pH was raised to 6.8 by carefully adding about twenty drops of 25% ammonium hydroxide. This was monitored using a pH electrode. The final yield was 980 ml The total solids content was 33%.

Composition Sample 105

Pre-Emulsion Preparation:

250 g deionized water by mixing with a magnetic stirrer. Then 23.3 g Sabowax SE40 (polyethylene glycol-40 stearate) cosmetic grade surfactant, were added to form a transparent viscous surfactant solution. In a 1 liter beaker were placed all of the monomers (see below). This monomer solution was poured into the surfactant solution and mixed to give a pre-emulsion. The pre-emulsion was kept for 20 hours in a refrigerator.

### Monomers/Oligomers:

55 g CN152 (Sartomer), Epoxy acrylate oligomer
64.5 g isobornyl monoacrylate,
20.7 g 2-(methacryloyloxy)ethyl acetoacetate (AAEM)—the cross-linker,

### Water, 350 g.

Polymerization

To the pre-emulsion 290 mg ammonium persulfate was added and it was stirred with a mechanical stirrer for 0.5 h. 100 ml of water were added to the reactor and the mechanical stirrer was set at 300 rpm. Then 100 ml of pre-emulsion were added. Nitrogen gas was purged through the mixture for 30 minutes. Some foam may be formed at this stage. The reactor was then placed in an oil bath and heated to 84° C. and then the remainder of the pre-emulsion was added to the reactor during two hours and 10 minutes using a peristaltic pump. To complete the polymerization, 29 mg of ammonium per sulfate were added and the temperature was raised to 95-97° C. for an additional hour. The heating was shut off and the contents of the reactor were cooled slowly with continuous stirring with the reactor remaining in the oil bath. The emulsion polymer product, a synthetic latex, was removed from the reactor and filtered through a synthetic non-woven cloth. No coagulation product was observed. The pH of the emulsion was 2.7. The pH was raised to 6.8 by carefully adding about twenty drops of 25% ammonium hydroxide. This was monitored using a pH electrode. The final yield was 440 mls and the total solids content was 32%.

### Example 5: Demonstration of Hair Straightening Effect

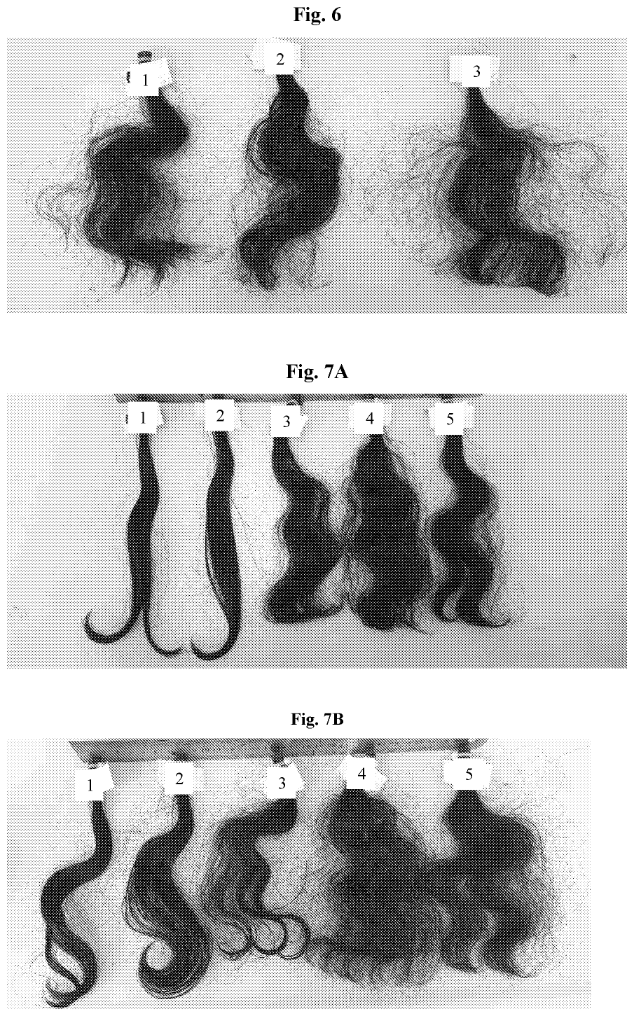

The experiments were performed on swatches of Brazilian hair type 2 (initial untreated swatches are presented in FIG. 6). The swatches were weighed and their end to end lengths were measured. The swatches were wetted and their length in straightened form was measured; all swatches at their extended state were about 24 cm long.

Compositions 101 and 105 were prepared as described in Example 4. Swatches 1 and 2 were treated with 15% dispersions of compositions 101 and 105 respectively as prepared without further additives. Swatches 3, 4 and 5 were treated with double distilled water only (controls).

Each swatch was treated in turn with formulation comprising 15% of composition (101 or 105) or water, combed and brushed and lightly dried with a domestic hair drier (FIG. 7A). After measuring the maximum swatch lengths, they were placed in a humidity chamber at 20° C. and 80% relative humidity for three days. The experimental arrangement and the results are summarized in Table 4.

TABLE 4
Summary of hair treatment with emulsions

| | Swatch | | Swatch | | Swatch | | Swatch | | Swatch | |
|---|---|---|---|---|---|---|---|---|---|---|
| | # 1 | | # 2 | | # 3 | | # 4 | | # 5 | |
| Treatment | 15% 101 in DDW | | 15% 105 in DDW | | DDW | | DDW | | DDW | |
| Weight | 3.38 | g | 3.36 | g | 3.45 | g | 3.45 | g | 3.45 | g |
| End | 17.2 | cm | 17.5 | cm | 16.5 | cm | 16.5 | cm | 16.5 | cm |
| to end | | | | | | | | | | |
| Length | | | | | | | | | | |
| Maximum | 24 cm +/− 0.3 mm | | 24 cm +/− 0.3 mm | | 24 cm +/− 0.3 mm | | 24 cm +/− 0.3 mm | | 24 cm +/− 0.3 mm | |
| length | | | | | | | | | 0.3 | mm |
| End | 21.2 | cm | 22.1 | cm | 20.5 | cm | 19.4 | cm | 19.9 | cm |
| to end | | | | | | | | | | |
| Length | | | | | | | | | | |
| after | | | | | | | | | | |
| Treatment | | | | | | | | | | |
| Length | 19.5 | cm | 20.5 | cm | 18.8 | cm | 18 | cm | 18.3 | cm |
| after | | | | | | | | | | |
| RH 80% | | | | | | | | | | |

| | | | |
|---|---|---|---|
| Aspect | Narrow | Narrow | Disorganized |
| feeling | & Wavy | & Wavy | |

After three days of exposure to extreme relative humidity of 80% (see FIG. 7B) the emulsion-treated swatches (swatches 1 and 2) had maintained more than 50% of their extension and preserved their narrow look. The swatches treated with water (3-5) were much shorter, broader and disorganized.

Although the present invention has been described herein above by way of preferred embodiments thereof, it can be modified, without departing from the spirit and nature of the subject invention as defined in the appended claims.

1-23. (canceled)

24. A hair styling stock emulsion comprising from about 5 wt % to about 55 wt % of at least one acrylic-based polymer comprising acrylic units that act as cross-linkers that will form bonds between the polymers, and from about 0.5 wt % to about 15 wt % of a non-ionic surfactant, in a cosmetically acceptable water carrier, wherein the polymer is derived from a combination of acrylic monomers and oligomers, the amount of said cross-linker units is from about 2.5% wt % to about 25 wt % of said acrylic-based polymer, wherein the non-ionic surfactant is selected from a pharmaceutical or cosmetic grade surfactant, and wherein the polymer chains undergo cross linking and thereby straighten the hair upon drying.

25. The hair styling stock emulsion of claim 24, wherein the acrylic monomers and acrylic oligomers are selected from the group consisting of monoacrylate oligomer, acrylate, methacrylate(s), isobornyl acrylate, butyl acrylate, lauryl methacrylate, 2-hydroxy-3-(prop-2-enoyloxy)propyl 2-methyl-2-propylhexanoate, and any combination thereof.

26. The hair styling stock emulsion of claim 24, wherein the polymer is derived from a combination of acrylic monomers and acrylic oligomers, said combination is selected from the group consisting of (i) a combination of a monoacrylate oligomer and isobornyl acrylate; (ii) a combination of a monoacrylate oligomer, isobornyl acrylate, and butyl acrylate; and (iii) a combination of a monoacrylate oligomer, isobornyl acrylate, and lauryl methacrylate.

27. The hair styling stock emulsion of claim 24, wherein the monoacrylate oligomer is 2-hydroxy-3-(prop-2-enoyloxy)propyl 2-methyl-2-propylhexanoate.

28. The hair styling stock emulsion of claim 24, wherein the cross-linker unit is 2-(methacryloyloxy)ethylacetoacetate (AAEM).

29. The hair styling stock emulsion of claim 24, wherein the emulsion is characterized by at least one of: (i) the amount of said cross-linkers is from about 5 to about 20 wt % of said acrylic-based polymer; (ii) the emulsion comprises from about 2 wt % to about 10 wt % of said surfactant; (iii) the cross-linker units, and moieties of acrylic monomer and acrylic oligomers collectively constitute from 85 to 100% of the polymer; (iv) the polymers undergo crosslinking upon drying; and (v) the emulsion further comprising a preservative.

30. The hair styling stock emulsion of claim 24, wherein the non-ionic surfactant is selected from the group consisting of ethoxylated and/or propoxylated alcohol or ester, Laureth-20, Laureth-21, Laureth-25, Laureth-30, Laureth-38, Laureth-40, tyloxapol, polysorbate (TWEEN), Disponil AFX 4060, Sabowax SE40, PPG-5-ceteth-20, PEG-20 sorbitan isostearate, alkoxylated alcohols, alkoxylated polyol esters, decyl glucoside, coco-glucoside, sucrose laurate, sorbitol, polyoxyethylene, sorbitan monolaurate, and any combination hereof.

31. The hair styling stock emulsion of claim 24, wherein the composition comprises from about 10 wt % to about 30 wt % of said polymer comprising cross-linkers in the amount of about 3 wt % to about 20 wt % of said polymer, and from about 2 wt % to about 10 wt % of said non-ionic surfactant.

32. The hair styling stock emulsion of claim 24, wherein the amount of solids of said composition is from about 20 wt % to about 55 wt %.

33. The hair styling stock emulsion of claim 24, wherein the glass transition temperature (Tg) of the polymer is from about 10° C. to about 50° C.

34. A hair care formulation comprising from about 2 wt % to about 50 wt % of the stock emulsion of claim 24.

35. The hair care formulation of claim 34, wherein the hair care is hair straightening.

36. The hair care formulation of claim 34, wherein the hair care formulation is selected from the group consisting of a spray, lotion, cream, foams, mousse, and gels.

37. The hair care formulation of claim 34, further comprising a cosmetically acceptable ingredient selected from the group consisting of preservatives, thickeners, viscosity modifiers, fatty alcohols, pH adjusting agents, buffers, coloring agents, guar gums, perfumes, PEGs, essential oils silicone fluid dimethicone, hydroxypropyl cellulose, carboxymethyl cellulose, and any combination thereof.

38. The hair care formulation of claim 35, wherein the formulation straightens the hair upon drying.

39. A method of styling hair comprising applying the formulation of claim 35 to the hair and drying the formulation.

40. The method of claim 39, wherein the hair styling comprises hair straightening.

41. The method of claim 39, further comprising the step of combing or brushing the hair to straighten it before drying.

42. The method of claim 39, wherein the formulation is applied to dry or wet hair.

43. The method of claim 39, wherein drying is selected from drying by heat or drying at room temperature.
